(19) United States
(12) Patent Application Publication
(10) Pub. No.: US 2013/0023849 A1
(43) Pub. Date: Jan. 24, 2013

(54) METHODS AND SYSTEMS FOR REDUCING VIRAL LOAD OF HEPATITIS C VIRUS IN HEMODIALYSIS PATIENTS

(71) Applicant: AETHLON MEDICAL, INC., San Diego, CA (US)

(72) Inventors: Richard H. Tullis, Encinitas, CA (US); Harold H. Handley, Encinitas, CA (US); James A. Joyce, San Diego, CA (US); Paul R. Duffing, San Diego, CA (US)

(73) Assignee: AETHLON MEDICAL, INC., San Diego, CA (US)

(21) Appl. No.: 13/626,748

(22) Filed: Sep. 25, 2012

Related U.S. Application Data
(63) Continuation of application No. 13/049,804, filed on Mar. 16, 2011, now abandoned.
Application No. 13/049,804 is a continuation of application No. PCT/US2009/057013, filed on Sep. 15, 2009.
(60) Provisional application No. 61/097,841, filed on Sep. 17, 2008.

(51) Int. Cl. A61M 1/34; A61M 1/16

(52) U.S. Cl. 604/500; 210/295; 210/194; 210/195.2; 210/638

(57) ABSTRACT

The present technology relates to methods and systems for the removal of pathogens and fragments thereof in hemodialysis patients. In particular, methods and systems are described where lectins can be used to remove the Hepatitis C virus and fragments thereof in hemodialysis patients, and to provide a sustained reduction in viral load.

### RELATED APPLICATIONS

This application is a continuation of U.S. Ser. No. 13/049,804 filed Mar. 16, 2011, which is a continuation of PCT International Application No. PCT/US2009/057013, filed Sep. 15, 2009 under the Patent Cooperation Treaty (PCT), which was published by the International Bureau in English, which designates the United States and claims the benefit of U.S. Provisional Patent Application No. 61/097,841, filed Sep. 17, 2008. The disclosures of each of the foregoing applications are hereby expressly incorporated by reference in their entireties.

### FIELD OF THE INVENTION

The present technology relates to the fields of medicine, virology, liver disease, immunology and biochemistry. In particular, methods and systems are described where lectins can be used to remove Hepatitis C virus and fragments thereof from the blood of hemodialysis patients, preferably providing a sustained reduction in HCV viral load.

### BACKGROUND

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

HCV is particularly prevalent in hemodialysis patients. Indeed, prevalence studies in developed countries indicate that up to 32.1% of hemodialysis patients have been exposed to HCV (Fabrizi F, et al: Epidemiology and clinical significance of hepatotropic infections in dialysis patients. Recent evidence. Minerva Urol, Nefrol, 56: 249-257, 2004; Saab S, et al.: Serum alanine aminotransferase in hepatitis C screening of patients on hemodialysis. Am. J. Kidney Dis. 37: 308-315, 2001; Schneeberger P M, et al: The prevalence and incidence of hepatitis C virus infections among dialysis patients in the Netherlands: A nationwide prospective study. J. Infect. Dis. 182: 1291-1299, 2000). The mechanism by which HCV establishes viral persistence and causes a high rate of chronic liver disease has not been thoroughly elucidated. It is not known how HCV interacts with and evades the host immune system. In addition, the roles of cellular and humoral immune responses in protection against HCV infection and disease have yet to be established.

Therapies to treat chronic Hepatitis C are limited. Patients can be treated with interferon-alpha, alone and in combination with other antiviral agents such as ribavirin. However, not all patients respond to treatment. For example, only 40%-50% of patients infected with the HCV genotype 1, the most common genotype in the United States, will respond and show a sustained virological response (defined as undetectable HCV RNA in the patient's blood 24 weeks after the end of treatment) to therapy. Moreover, such therapies can have serious side effects including anemia, cardiovascular events, and psychiatric problems. In addition, no vaccine exists to protect against HCV infection.

HCV therapy for dialysis patients may be an important means for reducing the risk of liver-related disease in patients that are on dialysis and after renal transplant. More than 341,319 patients in the United States required dialysis (U.S. Renal Data System <http://www.usrds.org>). The impetus to develop new therapies is further supported by findings that interferon is associated with an increased risk for renal allograft rejection when used after renal transplantation (Ozgur O. et al., Recombinant alpha-interfereon in renal allograft recipients with chronic hepatitis C, Nephrol. Dial. Transplant (1995) 10:2104-2106; Rostaing L. et al., Treatment of chronic hepatitis C with recombinant interferon-alpha in kidney transplant recipients, Transplantation (1995) 59:1426-1431).

Accordingly, there is an ongoing need to develop new therapies to treat patients infected with HCV, particularly hemodialysis patients infected with HCV.

### SUMMARY

Preferred embodiments of the present invention relate to methods, devices, systems and kits for reducing the viral load of Hepatitis C virus in blood from an individual infected with Hepatitis C. A preferred method combines a lectin affinity capture device with standard hemodialysis to remove HCV virus and fragments, reducing viral load. The combined treatment is continued for a period of time, typically on the same schedule as the patient's normal hemodialysis. In a preferred embodiment, the reduction in viral load is sustained for at least about a week following the last treatment with the combined hemodialysis and affinity capture device.

One embodiment of the invention is a method for producing a sustained reduction in the viral load of Hepatitis C virus (HCV) in blood from a patient undergoing hemodialysis comprising identifying a patient in need of hemodialysis that is infected with HCV; removing blood from the patient; passing the blood or portion thereof through a hemodialysis apparatus; providing a lectin affinity device comprising a processing compartment having lectin disposed within the processing chamber, where the lectin binds HCV viral particles in the blood, and where the device is configured to retain the lectin and bound HCV viral particles in the processing compartment; transferring the blood or a portion thereof into the compartment such that the HCV viral particles contact the lectin and are bound thereto; removing the blood or portion thereof from the chamber, and returning the blood or portion thereof to the patient. In some embodiments the method is repeated for about 1 to 6 hours a day, for at least 3 days a week. In some embodiments the method is discontinued after a period of time not less than about 1 week, where the method reduces the viral load, and where the viral load is reduced for a period of at least about 1 week following the discontinuation of the method. In some embodiments the portion of blood is the plasma.

Another embodiment is an improved hemodialysis method for patient's undergoing hemodialysis who are infected with HCV, the improvement comprising passing the patient's blood through an lectin affinity device during the hemodialysis, where the lectin affinity device comprises a processing compartment having lectin disposed within the processing chamber, where the lectin binds HCV viral particles in the blood, and where the device is configured to retain the lectin and bound HCV viral particles in the processing compartment.

In some embodiments, methods for reducing the viral load of Hepatitis C virus in blood from an individual infected with Hepatitis C include providing a lectin affinity device containing a processing chamber that includes lectin disposed within the processing chamber, in which the lectin binds viral particles in the blood and traps the viral particles in the processing chamber; transferring the blood into the chamber such that viral particles contact the lectin and are bound to the lectin; removing the blood from the chamber, and optionally repeating the transferring and removing steps.

In some embodiments the individual is a hemodialysis patient. In further embodiments, the hemodialysis patient is undergoing hemodialysis.

In some embodiments, methods for reducing the viral load of Hepatitis C virus in blood from an individual infected with Hepatitis C include reinfusing the blood into the individual.

In some embodiments, the blood is exposed to the lectin for no longer than 360 minutes.

In some embodiments, the transferring and removing step is repeated as often as required to remove at least 50% of the viral load from the blood. In some embodiments, the transferring and removing step is repeated as often as required until the remaining viral load is no greater than 1×10⁵ copies/ml of HCV RNA. In some embodiments, the transferring and removing is repeated until a volume of blood approximately equal to the total blood volume of the individual has been exposed to the lectin.

In some embodiments, methods for reducing the viral load of Hepatitis C virus in blood from an individual infected with Hepatitis C include methods where the lectin is selected from a group consisting of Galanthus nivalis agglutinin (GNA), Narcissus pseudonarcissus agglutinin (NPA), cyanovirin (CVN), Concanavalin A, Griffithsin and mixtures thereof. In particular embodiments, the lectin is GNA. In further embodiments, the lectin binds to a HCV viral coat protein or a fragment thereof.

In certain methods, the chamber further includes one or more porous hollow fiber membranes in the chamber, in which lectin is disposed within an extralumenal space of the chamber proximate to an exterior surface of the membranes, and in which the lectin binds the viral particles and traps them in the extralumenal space; such methods further include passing the blood through the hollow fiber membranes; and collecting pass-through blood. Further embodiments include repeating the passing and collecting steps with the pass-through blood to further reduce the amount of the viral load in the pass-through blood. Further embodiments include methods in which the porous membranes allow passage of intact viral particles through the pores and exclude substantially all blood cells from passing through the pores.

Some methods for reducing the viral load of Hepatitis C virus in blood from an individual infected with Hepatitis C include a processing chamber further containing a porous membrane, in which the membrane is configured such that the porous membrane allows passage of viral particles through the pores such that the viral particles contact the lectin, and the porous membrane excludes substantially all blood cells from passing through the pores, such that the blood cells do not contact the lectin. In such methods, the membrane has pores less than about 700 nm in diameter. Also, the membrane can be a porous hollow fiber membrane. The membranes have an inside diameter of about 0.3 mm and an outside diameter of about 0.5 mm.

In some embodiments, the lectin is attached to a substrate. In such embodiments, the substrate is selected from the group consisting of agarose, aminocelite, resins, silica, and proteins. In further embodiments, the substrate is a silica selected from the group consisting of glass beads, sand, and diatomaceous earth. In some embodiments, the substrate is a polysaccharide selected from the group consisting of dextran, cellulose and agarose. In some embodiments, the substrate is a protein comprising gelatin. And in some embodiments, the substrate is a plastic selected from the group consisting of polystyrenes, polysulfones, polyesters, polyurethanes, polyacrylates and their activated and native amino and carboxyl derivatives. In certain embodiments, the lectin is linked to the substrate by a linker. The linker can be selected from the group consisting of glutaraldehyde, C₂ to C₁₈ dicarboxylates, diamines, dialdehydes, dihalides, and mixtures thereof.

In addition to the foregoing methods, systems for reducing the viral load of Hepatitis C virus in blood from an individual infected with Hepatitis C are also described. Such systems include a lectin affinity device containing a processing chamber having lectin disposed within the processing chamber, in which the lectin binds viral particles in the blood and traps the viral particles in the processing chamber; and a dialysis apparatus. In certain embodiments, the individual is a hemodialysis patient. In some embodiments, the hemodialysis patient is undergoing hemodialysis. In particular embodiments, the lectin affinity device and the dialysis apparatus are utilized simultaneously.

In some systems, the lectin affinity device and the dialysis apparatus are connected to establish an extracorporeal circulation system with the individual. In such embodiments, the lectin affinity device and the dialysis apparatus are connected in series, or are connected in parallel.

In addition to the foregoing methods and systems, also described herein are kits for reducing the viral load of Hepatitis C virus in blood from an individual infected with Hepatitis C. Such kits include a lectin affinity device containing a processing chamber having lectin disposed within the processing chamber in which the lectin binds viral particles in the blood and traps the viral particles in the processing chamber; and also includes instructions for use of the lectin affinity device.

### BRIEF DESCRIPTION OF THE DRAWINGS

FIG. 1 is a schematic illustration of a longitudinal cross section of an embodiment of an affinity cartridge.

FIG. 2 is a schematic illustration of a horizontal cross section at plane 2 in FIG. 1.

FIG. 3 is an illustration of a hollow fiber from FIG. 2.

FIG. 4 is a graph of the average HCV viral load (IU/ml) following dialysis alone (Control), following treatment with a lectin affinity device plus dialysis (HP+dialysis), and one week later (Final).

### DETAILED DESCRIPTION

Embodiments of the present invention relate to methods, systems and kits for reducing the viral load of Hepatitis C virus in blood from an individual infected with Hepatitis C. In particular, methods, systems and kits utilizing lectins to reduce the viral load of Hepatitis C in hemodialysis patients are described herein. A preferred method combines a lectin affinity capture device with standard hemodialysis to remove HCV virus and fragments, reducing viral load in the blood of the infected hemodialysis patient. The combined treatment is continued for a period of time (1-4 weeks), typically on the same schedule as the patient's normal hemodialysis (e.g., once a day for 2-4 hours, 3 days a week). In a preferred embodiment, the reduction in viral load is sustained for at least about a week following the last treatment with the combined hemodialysis and affinity capture device.

Some embodiments described herein include using the GNA HEMOPURIFIER® to reduce the HCV viral load of HCV-infected dialysis patients.

Previous studies have shown that a GNA HEMOPURIFIER® similar to the device used in Example 1, can bind up to 5×10⁹ HCV viral particles in a 4 hour session. Thus, it would be expected that approximately 55% of viral particles would be cleared from an HCV-infected individual by the GNA HEMOPURIFIER® in a 4 hour session, leaving approximately 4.3×10¹⁹ HCV viral particles circulating in the body of the individual.

However, HCV can replicate at a high rate. Indeed, HCV can replicate in an infected individual at a rate of up to 1.7×10¹¹ viral particles in 4 hours (10¹² virus particles in 24 hours). Thus, an infected individual may produce in 4 hours approximately 9 times more viral particles than the GNA HEMOPURIFIER® would be expected to remove from an individual, taking into account the body's natural clearance. Therefore, it would be expected that in a series of sessions using the GNA HEMOPURIFIER®, HCV-infected individuals would show no significant change in the total HCV viral load.

However, in HCV-infected hemodialysis patients using the GNA HEMOPURIFIER® a significant level of HCV clearance can be observed, and importantly, the reduced viral load is unexpectedly maintained for at least a week after treatment.

The following description illustrates embodiments of the present technology. In some embodiments described herein can utilize methods and devices found in pending PCT Patent Application No. PCT/US2008/063946, entitled “Device and Method for Purifying Virally Infected Blood” filed May 16, 2008, the disclosure of which is incorporated herein in its entirety by reference.

The term “blood” as used herein can include blood components, or portions thereof, for example, plasma.

The term “hemodialysis patient” as used herein can include individuals and/or patients with renal failure. Renal failure includes acute renal failure and chronic renal failure. Individuals and/or patients with renal failure may need renal replacement therapy. Renal replacement therapies include dialysis or kidney transplant.

The term “undergoing hemodialysis” as used herein can include the period of time when a hemodialysis patient is utilizing a dialysis apparatus.

The term “viral load” as used herein refers to the amount of viral particles or fragments thereof in a biological fluid, such as blood or plasma. “Viral load” encompasses all viral particles, infectious, replicative and non-infective, and fragments thereof. Therefore, viral load represents the total number of viral particles and/or fragments thereof circulating in the biological fluid. Viral load can therefore be a measure of any of a variety of indicators of the presence of a virus, such as viral copy number per unit of blood or plasma, units of viral proteins or fragments thereof per unit of blood or plasma, or HCV RNA copies per milliliter of blood or plasma. RNA copies can be measured using techniques well known in the art, for example, using quantative RT-PCR.

Viral load correlates with the likelihood of a response to other viral therapies. Therefore, reducing viral load can improve the effectiveness of other therapies. In some embodiments, the methods and devices described herein are combined with existing treatments for HCV, including, but not limited to drug therapies, interferon-alpha, alone and in combination with other antiviral agents such as ribavirin.

The term “plaque forming units” or “pfu” as used herein refers to the amount of infectious virus particles in a biological fluid, such as blood or plasma. One plaque forming unit is formally equivalent to one infectious virus particle. A skilled artisan would recognize that viral plaque forming units are more critical to reduce than viral load. In a preferred embodiment of the present invention, pfu/ml is reduced more efficiently than reducing viral load.

One skilled in the art would recognize that there are several ways to determine the number of plaque forming units in a particular sample. See, e.g., Lee H, and Jeong, Y S (2004) Comparison of Total Culturable Virus Assay and Multiplex Integrated Cell Culture-PCR for Reliability of Waterborne Virus Detection. Appl Environ Microbiol. 2004 June; 70(6): 3632-3636. In one particular assay, cells are grown on a flat surface until they form a monolayer of cells covering a bottle or dish. They are then infected with the target sample, or a particular dilution thereof. A plaque is produced when a virus particle infects a cell, replicates, and lyses, killing the cell. Surrounding cells are infected by the newly replicated virus and they too are killed. This process can repeat several times, such that sufficient numbers of neighboring cells are infected and lysed to form a cell-free hole within the monolayer of cells. The cells can be stained with a dye which stains only living cells. The dead cells in the plaque do not stain and appear as unstained areas on a colored background. Each plaque is the result of infection of one cell by one virus followed by replication and spreading of that virus. However, viruses and fragments that do not kill cells can not produce plaques and can contribute to the viral load without affecting the pfu count.

The term “high mannose glycoprotein” as used herein for the purpose of the specification and claims refers to glycoproteins having mannose-mannose linkages in the form of α-1->3 or α-1->6 mannose-mannose linkages. Some examples of lectins which bind glycoproteins including high mannose glycoproteins include, without limitation, Galanthus nivalis agglutinin (GNA), Narcissus pseudonarcissus agglutinin (NPA), cyanovirin (CVN), Concanavalin A, Griffithsin and mixtures thereof. In addition to lectins, also contemplated are other carbohydrate binding proteins, including adhesins, selectins, cell adhesion molecules, and mannose binding protein (MBP).

The term “exposed,” as used herein in the context of blood being exposed to any type of lectin-containing substrate, refers to any virus-containing portion of blood contacting a lectin-containing substrate. In some embodiments, the blood is exposed to the lectin-containing substrate for a specific amount of time. Exposure of the blood to the lectin-containing substrate, as used herein, refers to the total amount of time the blood is exposed to the lectin-containing substrate and not the amount of time blood is processed through the device.

The time of exposure is a function of the flow rate and the capacity of the lectin-containing substrate. For example, if the flow rate of a device is 10 ml/min and the capacity of the device is 10 ml, then running unprocessed blood for 30 minutes would expose 300 ml of blood to the lectin-containing substrate for 1 minute. For further illustration, if 30 ml of blood were recirculated over a device with the same flow rate and same capacity for 30 minutes, then the 30 ml of blood would be exposed to the lectin-containing substrate for 10 minutes. In some embodiments, the blood is exposed to a lectin-containing substrate is, is about, is less than, is less than about, is more than, is more than about, 600 minutes, 550 minutes, 500 minutes, 490 minutes, 480 minutes, 470 minutes, 460 minutes, 450 minutes, 440 minutes, 430 minutes, 420 minutes, 410 minutes, 400 minutes, 390 minutes, 380 minutes, 370 minutes, 360 minutes, 350, 340 minutes, 330 minutes, 320 minutes, 310 minutes, 300 minutes, 290 minutes, 280 minutes, 270 minutes, 260 minutes, 250 minutes, 240 minutes, 230 minutes, 220 minutes, 210 minutes, 200 minutes, 190 minutes, 180 minutes, 170 minutes, 160 minutes, 150 minutes, 140 minutes, 130 minutes, 120 minutes, 110 minutes, 100 minutes, 90 minutes, 80 minutes, 70 minutes, 60 minutes, 50 minutes, 40 minutes, 30 minutes, 20 minutes, 19 minutes, 18 minutes, 17 minutes, 16 minutes, 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes, or 1 minute. In other embodiments, the time the blood is exposed to a lectin-containing substrate is a range defined by any two times recited above.

In some embodiments, the flow rate through the device is about 60 ml/min to about 400 ml/min. In some embodiments, the flow rate through the device is about 250 ml/min to about 400 ml/min. In some embodiments, the flow rate is, is about, is less than, is less than about, is more than, is more than about, 600 ml/min, 550 ml/min, 500 ml/min, 490 ml/min, 480 ml/min, 470 ml/min, 460 ml/min, 450 ml/min, 440 ml/min, 430 ml/min, 420 ml/min, 410 ml/min, 400 ml/min, 390 ml/min, 380 ml/min, 370 ml/min, 360 ml/min, 350 ml/min, 340 ml/min, 330 ml/min, 320 ml/min, 310 ml/min, 300 ml/min, 290 ml/min, 280 ml/min, 270 ml/min, 260 ml/min, 250 ml/min, 240 ml/min, 230 ml/min, 220 ml/min, 210 ml/min, 200 ml/min, 190 ml/min, 180 ml/min, 170 ml/min, 160 ml/min, 150 ml/min, 140 ml/min, 130 ml/min, 120 ml/min, 110 ml/min, 100 ml/min, 90 ml/min, 80 ml/min, 70 ml/min, 60 ml/min, 50 ml/min, 40 ml/min, 30 ml/min, 20 ml/min, 19 ml/min, 18 ml/min, 17 ml/min, 16 ml/min, 15 ml/min, 14 ml/min, 13 ml/min, 12 ml/min, 11 ml/min, 10 ml/min, 9 ml/min, 8 ml/min, 7 ml/min, 6 ml/min, 5 ml/min, 4 ml/min, 3 ml/min, 2 ml/min, or 1 ml/min, or a range defined by any two of these values. In some embodiments, the capacity of the device is 40 ml. Also contemplated are devices where the capacity is about, is less than, is less than about, is more than, is more than about, 600 ml, 550 ml, 500 ml, 490 ml, 480 ml, 470 ml, 460 ml, 450 ml, 440 ml, 430 ml, 420 ml, 410 ml, 400 ml, 390 ml, 380 ml, 370 ml, 360 ml, 350 ml, 340 ml, 330 ml, 320 ml, 310 ml, 200 ml, 290 ml, 280 ml, 270 ml, 260 ml, 250 ml, 240 ml, 230 ml, 220 ml, 210 ml, 200 ml, 190 ml, 180 ml, 170 ml, 160 ml, 150 ml, 140 ml, 130 ml, 120 ml, 110 ml, 100 ml, 90 ml, 80 ml, 70 ml, 60 ml, 50 ml, 40 ml, 30 ml, 20 ml, 19 ml, 18 ml, 17 ml, 16 ml, 15 ml, 14 ml, 13 ml, 12 ml, 11 ml, 10 ml, 9 ml, 8 ml, 7 ml, 6 ml, 5 ml, 4 ml, 3 ml, 2 ml, or 1 ml, or a range defined by any two of these values.

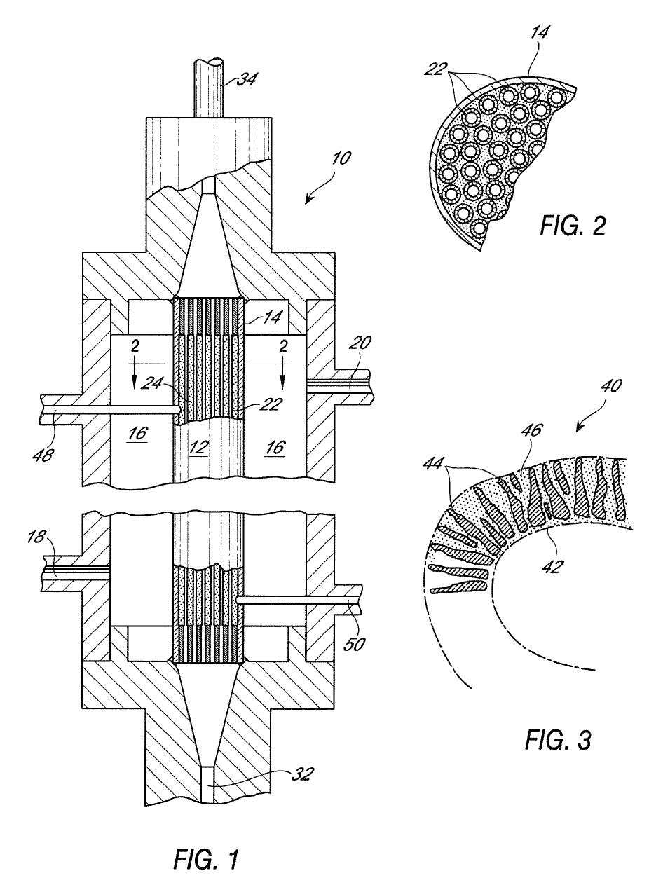

Some embodiments include methods utilizing an affinity cartridge such as the device illustrated in FIG. 1 and described below in greater detail. Devices of this general type are disclosed in U.S. Pat. Nos. 4,714,556, 4,787,974 and 6,528,057, the entire disclosures of which are incorporated herein by reference. In such devices, blood can be passed through the lumen of a hollow fiber membrane, wherein lectins are located in the extralumenal space of the cartridge, which form a means to accept and immobilize viruses and toxic and/or infectious fragments thereof. Thus, the device retains intact virions and viral glycoproteins bound by lectin while allowing other blood components to pass through the lumen.

In preferred embodiments, methods and systems described herein include removal of HCV. In some embodiments, a virus can include one or more of the following types: enveloped virus, Category A enveloped virus, ebola, marburg, smallpox, lassa, dengue, rift valley, west nile, influenza (e.g., H5N1), measles, mumps, viral encephalitis (e.g. Japanese encephalitis), monkeypox, camelpox, vaccinia, HIV, HCV, hepatitis virus, human cytomegalovirus (HCMV), distemper, swine pox, swine flu, siv, fiv, bird flu, sin nombre, yellow fever, herpes, SARS, sendai. Other embodiments include one or more viruses from the families of retroviridae, poxyiridae paramyxoviridae (e.g., measles, mumps, sendai), orthomyxoviridae (e.g., bird flu, influenza), filoviridae (e.g., ebola, marburg), coronaviridae (e.g., SARS, encephalomyelitis), herpesviridae (e.g., herpes simplex, HCMV), rhabdoviridae (e.g., varicella stomatitis, rabies), and togavirus (e.g., rubella, semliki). Further embodiments include any lectin-binding virus, namely, any virus or fragment thereof which binds to lectin or is bound by lectin.

In certain embodiments, a virus may not include at least one or more of the viruses selected from the group consisting of ebola, marburg, smallpox, lassa, dengue, rift valley, west nile, influenza (e.g., H5N1), measles, mumps, viral encephalitis (e.g. Japanese encephalitis), monkeypox, camelpox, vaccinia, HIV, HCV, hepatitis virus, human cytomegalovirus (HCMV), distemper, swine pox, swine flu, siv, fiv, distemper, bird flu, sin nombre, yellow fever, herpes, SARS, or sendai.

One embodiment of an affinity device, described in detail below with reference to FIGS. 1-3, includes multiple channels of hollow fiber membrane that forms a filtration chamber. An inlet port and an effluent port are in communication with the filtration chamber. The membrane is preferably an anisotropic membrane with the tight or retention side facing the bloodstream. The membrane is formed of any number of polymers known to the art, for example, polysulfone, polyethersulfone, polyamides, polyimides, and cellulose acetate. In other embodiments, the porous membrane is a sheet, rather than a channel. The sheet can be flat, or in some other configuration, such as accordion, concave, convex, conical, etc., depending on the device. In some embodiments, the membrane has pores with a mean diameter of, of about, of less than, of less than about, of more than, of more than about, 1950 nm, 1900 nm, 1850 nm, 1800 nm, 1750 nm, 1700 nm, 1650 nm, 1600 nm, 1550 nm, 1500 nm, 1450 nm, 1400 nm, 1350 nm, 1300 nm, 1250 nm, 1200 nm, 1150 nm, 1100 nm, 1050 nm, 1000 nm, 950 nm, 900 nm, 850 nm, 800 nm, 750 nm, 700 nm, 650 nm, 640 nm, 630 nm, 620 nm, 610 nm, 600 nm, 590 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 90 nm, or 85 nm, which will allow passage of intact viruses and viral particles and fragments (e.g., HCV of 50 nm, Rous Sarcoma Virus virions of 80 nm diameter), but not most blood cells. In other embodiments, the membrane has pores in a range between any two pore diameters recited above.

In particular embodiments, the membrane can have pores 200-500 nm in diameter, more preferably, the pore size is 600 nm, which will allow passage of intact viruses and viral particles and fragments (e.g., HIV virions of 110 nm diameter), but not most blood cells (red blood cells 10,000 nm diameter, lymphocytes 7,000-12,000 nm diameter, macrophages 10,000-18,000 nm diameter, thrombocytes 1000 nm). Optionally, by selecting a pore size that is smaller than the diameter of blood cells, the membrane excludes substantially all blood cells from passing through the pores and entering the extrachannel or extralumenal space of the device that contains the lectin. In some embodiments, a pore size is selected that is smaller than only some blood cell types.

A diagram of one embodiment of the device is shown in FIG. 1. The device comprises a cartridge 10 comprising a blood-processing chamber 12 formed of interior glass or plastic wall 14. Around chamber 12 is an optional exterior chamber 16. A temperature controlling fluid can be circulated into chamber 16 through port 18 and out of port 20. The device includes an inlet port 32 for the blood and an outlet port 34 for the effluent. The device also provides one or more ports 48 and 50, for accessing the extrachannel or extralumenal space in the cartridge. FIG. 2 is a schematic illustration of a horizontal cross section at plane 2 in FIG. 1. As shown in FIGS. 1 and 2, chamber 12 contains a plurality of membranes 22. These membranes preferably have a 0.3 mm inside diameter and 0.5 mm outside diameter. In some embodiments, the outside or inside diameter is 0.025 mm to 1 mm more preferably 0.1 to 0.5 mm more preferably 0.2 to 0.3 mm, as close to the outside diameter as allowed to minimize flow path length while still providing structural integrity to the fiber. FIG. 3 is a cross sectional representation of a channel 22 and shows the anisotropic nature of the membrane. As shown in FIG. 3, a hollow fiber membrane structure 40 is preferably composed of a single polymeric material which is formed into a tubular section comprising a relatively tight plasmapheresis membrane 42 and relatively porous exterior portion 44 in which can be immobilized lectins 46. During the operation of the device, a solution containing the lectins is loaded on to the device through port 48. The lectins are allowed to immobilize to the exterior 22 of the membrane in FIG. 2. Unbound lectins can be collected from port 50 by washing with saline or other solutions. Alternatively, the lectins can be bound to a substrate which is loaded into the extrachannel or extralumenal space, either as a dry substance (e.g. sand), or in solution or slurry.

In another embodiment, the device comprises a processing chamber having lectin disposed within the processing chamber, wherein said lectin binds viral particles or fragments in the blood or plasma, and traps them in the processing chamber. The blood or plasma can directly contact the lectins. In other embodiments, the device has a porous membrane which divides the chamber into one or more portions, such that the lectin is located in only a portion of the chamber. The preferred device utilizes hollow channel fiber membranes, but one or more sheets of membranes that divide the chamber are also contemplated. Where a membrane is used, the blood or plasma is filtered by the membrane, such that some portion of the blood or plasma is excluded from the portion of the chamber containing the lectin (e.g., blood cells or other large cells which cannot pass through the pores of the membrane).

In some embodiments, a device and method for reducing the viral load or pfu/ml in the blood or plasma by a therapeutically effective amount are provided. As used herein, the term “therapeutically effective amount” refers to a viral load or pfu/ml in the blood or plasma that halts or slows the progression of the infection, and slows or prevents the worsening of symptoms associated with the infection, and preferably improves or eliminates the infection or symptoms thereof. In some cases, reducing viral load or pfu/ml by or to a “therapeutically effective amount” can allow an infected individual's immune system to maintain or reduce the viral load or pfu/ml without further intervention. In some embodiments, “therapeutically effective amount” is an amount sufficient to render another treatment (e.g. a drugs, retroviral therapy, etc.) effective, or more effective. The “therapeutically effective amount” varies with different viruses and individuals, but can be readily determined by a skilled artisan. For example, for HCV, some studies indicate that asymptomatic viral loads in plasma can vary between 1×10² copies/ml and 5×10⁷ copies/ml of HCV RNA (Ulrich et al., Detection, semiquantitation, and genetic variation in hepatitis C virus sequences amplified from the plasma of blood donors with elevated alanine aminotransferase (1990) 86: 1609-1614), however, under some circumstances an asymptomatic viral loads may not represent a therapeutically effective amount; for HIV infection, current antiviral treatments have a target level of no greater than about 1000 copies/ml; and for Ebola, infected monkeys are said to resolve disease on their own if the count can be reduced below 50,000 copies/ml (as measured by quantitative RT-PCR).

As illustrated in Table 1, the copies of virus per ml, varies from virus to virus. Just as the average viremia before clearance varies between viruses, so does the desired viral load or pfu/ml after clearance.

TABLE 1

| | Viremia (copies per ml plasma) | | | | |
|---|---|---|---|---|---|
| Virus | Max | Mean | Survivable | Lethal | Ref |
| Crimean Congo hemorrhagic fever | | 7.7 × 10⁵ | | | 1 |
| Dengue fever | 1.5 × 10⁷ | 4.0 × 10⁷ | 4.0 × 10⁷ | | 5, 11 |
| Dengue fever-febrile | | 8 × 10⁵ | | | 1 |
| Dengue fever-defevrescent | | | | | 5 |
| Dengue hemorrhagic fever | 2 × 10⁹ | 3.2 × 10⁸ | 4.0 × 10⁷ | 3.2 × 10⁸ | 11 |
| Dengue hemorrhagic fever-febrile | | 1.5 × 10⁶ | | | |
| Dengue hemorrhagic fever- | | 4.3 × 10⁵ | | | |
| defevrescent | | | | | |
| Ebola | 1 × 10⁹ | | 1 × 10⁷ | 6.9 × 10⁸ | 1, 7 |
| Hepatitis C virus | | 3.2 × 10⁶ | | | 15 |
| HIV | 2 × 10⁶ | 2 × 10⁴ | 1 × 10³ | | 15, 16 |
| Lassa virus | 4 × 10⁹ | 7 × 10⁶ | | 4 × 10³ | 1, 8 |
| Rift Valley fever | 1 × 10⁹ | | | | 13 |
| Sin Nombre | | 1.3 × 10⁶ | 6.3 × 10⁵ | 5.0 × 10⁶ | 4 |
| Smallpox (Vaccinia) | 2 × 10⁵ | | | | 12 |
| West Nile Virus | 1 × 10⁷ | | | | 10 |
| Yellow fever | 1 × 10⁶ | 4 × 10⁵ | | | 1 |

References
1. Gunther, S (2002) J. Clinical Microbiology, 40 (7): 2323-2330.
2. Drosten, et al NEJM 348 (20): 1967-76, 2003
3. Zwiers, Miller, Baker, Kulesh, Jahrling and Huggins (USAMRIID)
4. Terajima, et al (1999) J Infect Dis 180: 2030.
5. Wang, WK et al (2003) Virology 20: 330.
6. Sanchez, et al (2004) J, Virol 18: 10370
7. Towner et al (2004) J. Virol 78: 4330
8. McCormick et al (1986) NEJM 314: 20
9. Schmitz et al (2002) Microbes Infect 4(1): 43-50
10. Paddock et al. (2006) CID 2006: 42 (June 1) 1527
11. Vaughn et al (2000) J Infect Dis 181: 2-9
12. Sharon et al (2003) JAMA Jun. 25, 2003 289 (24) 3295
13. Niklasson et al (1983) Journal of Clinical Microbiology 1026-1031 17(6)
14. Monath et al (2001) Lancet Infectious Diseases 1: 11-20
15. National Genetics Institute.

In some embodiments, a “therapeutically effective amount,” or the desired viral load or pfu/ml after clearance is, is about, is less than, is less than about, is more than, is more than about 1×10⁹ pfu/ml, 5×10⁸ pfu/ml, 1×10⁸ pfu/ml, 5×10⁷ pfu/ml, 1×10⁷ pfu/ml, 5×10⁶ pfu/ml, 1×10⁶ pfu/ml, 500,000 pfu/ml, 450,000 pfu/ml, 400,000 pfu/ml, 350,000 pfu/ml, 300,000 pfu/ml, 250,000 pfu/ml, 200,000 pfu/ml, 150,000 pfu/ml, 100,000 pfu/ml, 90,000 pfu/ml, 80,000 pfu/ml, 70,000 pfu/ml, 60,000 pfu/ml, 50,000 pfu/ml, 45,000 pfu/ml, 40,000 pfu/ml, 35,000 pfu/ml, 30,000 pfu/ml, 25,000 pfu/ml, 20,000 pfu/ml, 15,000 pfu/ml, 10,000 pfu/ml, 9000 pfu/ml, 8000 pfu/ml, 7000 pfu/ml, 6000 pfu/ml, 5000 pfu/ml, 4000 pfu/ml, 3000 pfu/ml, 2000 pfu/ml, 1000 pfu/ml, 900 pfu/ml, 800 pfu/ml, 700 pfu/ml, 600 pfu/ml, 500 pfu/ml, 450 pfu/ml, 400 pfu/ml, 350 pfu/ml, 300 pfu/ml, 250 pfu/ml, 200 pfu/ml, 190 pfu/ml, 180 pfu/ml, 170 pfu/ml, 160 pfu/ml, 150 pfu/ml, 140 pfu/ml, 130 pfu/ml, 120 pfu/ml, 100 pfu/ml, 95 pfu/ml, 90 pfu/ml, 85 pfu/ml, 80 pfu/ml, 75 pfu/ml, 70 pfu/ml, 65 pfu/ml, 60 pfu/ml, 55 pfu/ml, 50 pfu/ml, 45 pfu/ml, 40 pfu/ml, 35 pfu/ml, 30 pfu/ml, 25 pfu/ml, 20 pfu/ml, 15 pfu/ml, 10 pfu/ml, 0 pfu/ml. In some embodiments, the desired pfu/ml after clearance is a range defined by any two of the preceding numbers.

In some embodiments, the device is connected to an individual wherein the inlet port of the device is linked to the individual's vascular system, allowing blood to flow from the individual into the device, optionally with the assistance of a pump. In other embodiments, the blood from the individual is filtered or separated, allowing only the virus containing component to be exposed to a lectin-containing membrane. In some embodiments, the outlet port is also linked to the individual's vasculature to allow the effluent blood to be reinfused into the individual. In one embodiment, the purified plasma is mixed with the cellular component before being reinfused into the individual. In another embodiment, the cellular component of the blood is reinfused into the individual separate from the effluent plasma.

In a preferred embodiment, the affinity capture device is used while an individual undergoes hemodialysis. During dialysis, an individual is connected to a dialysis apparatus as is well known in the art. The inlet of a dialysis apparatus is connected to a patient, such that blood flows from the patient to the inlet of the dialysis device and through a dialysis cartridge; the outlet of the dialysis apparatus is connected to the individual, such that blood flows from the outlet of the dialysis device to the patient. In such embodiments, the device and a dialysis apparatus can form a continuous circuit with the patient, where blood and/or plasma enter the inlet of the device and the inlet dialysis apparatus, and re-enters the patient from the outlet of the affinity device and the outlet dialysis apparatus.

In a preferred embodiment, the affinity device and dialysis apparatus are in series. The affinity device can be upstream or downstream of the dialysis apparatus. In embodiments where more than one affinity device may be used, the affinity device may be upstream and downstream of the dialysis apparatus. Similarly, where more than one dialysis apparatus may be used, the dialysis apparatus may be upstream and downstream of the affinity device. In a preferred embodiment, the affinity capture device is upstream of the dialysis cartridge. This configuration permits the dialysis cartridge to filter from the blood any lectin and/or substrate (e.g. silica) that inadvertently escapes from the affinity capture cartridge prior to returning the blood to the individual.

In some embodiments, the affinity device and dialysis apparatus can be in parallel. Here, blood and/or plasma from the individual can enter either the inlet of the device or the inlet of the dialysis apparatus. Blood and/or plasma can then flow from the outlet of the device and the outlet of the dialysis apparatus to the individual.

In some embodiments, the affinity capture device is used every time the patient undergoes dialysis, which is typically once a day, every other day for a total of three days a week, for about 2, 3, 4, 5, or 6 hours each treatment. In some embodiments, the treatment is one, two, three or more times a day, and occurs 1, 2, 3, 4, 5, 6, or 7 times a week. The combined treatment with dialysis plus affinity capture can be continued for a period of time that is, is about, is at least, or is at least about, 1 day, 2 days, 3 days, 4 days, 5 days, or 6 days, 1 week, 2 weeks, 3 weeks, 4 weeks, 5 weeks, or 6 weeks, 1 month, 2 months, 3 months, 4 months, 5 months, or 6 months, 0.5 year, 1 year, 1.5 years, 2 years, 3 years, 4 years, 5 years or more years, or a range defined by any two of the proceeding values. In a preferred embodiment, the combined treatment is for a period of about 1 to 30 days, more preferably 3 to 14 days. In some embodiments, the HCV viral load is monitored by sampling the patient's blood on a regular basis, for example, 1, 2, 3 or more times in 1 day, 2 days, 3 days, 4 days, 5 days, 6 days or 7 days, 1 week, 2 weeks, 3 weeks, 4 weeks, or 5 weeks, or 1 month, 2 months, 3 months, 4 months, 5 months, 6 months, 7 months, 8 months, 9 months, 10 months, 11 months or 12 months. The combined treatment is continued until the viral load is reduced to a predetermined level, such as a therapeutically effective level.

In some embodiments, combined treatment results in a reduction in viral load that is maintained for a period of time following the cessation of combined treatment and return to kidney dialysis only. In some embodiments, the reduction in viral load is sustained for a period that is, is about, is at least, or is at least about, 3 days, 4 days, 5 days, or 6 days, 1 week, 2 weeks, 3 weeks, 4 weeks, 5 weeks, or 6 weeks, 1 month, 2 months, 3 months, 4 months, 5 months, or 6 months, 0.5 year, 1 year, 1.5 years, 2 years, 3 years, 4 years, 5 or more years, or a range defined by any two of the proceeding values. In a preferred embodiment, the reduction is sustained for at least about 1 week.

Some embodiments can include a device where affinity capture cartridge and dialysis cartridge are combined into a single unit.

In some embodiments, a volume equal to the total blood volume of the individual being treated is allowed to circulate at least once through the device. This does not necessarily mean that all of the blood in the individual passes through the device. As the blood is filtered and recirculated into the individual's blood stream, it is diluted by blood already present in the individual's blood stream. As such, it would be difficult to determine when all of the blood in the individual is circulated through the device. However, it can be determined when a volume equal to all of the individual's blood has been treated. Accordingly, the volume equal to the total blood volume of the individual being treated is defined as the total volume of blood run through the device being approximately equal to the estimated total blood volume present in the bloodstream of the individual being treated. For humans, the total blood volume for an average adult male weighing approximately 70 kg is between approximately 4 L and 5 L, (approximately 66 ml/kg) and the total volume of blood for an average adult female weighing approximately 50 kg is between approximately 3.0 L and 3.5 L (approximately 60 ml/kg). In some embodiments, a multiple of the total blood volume is treated. This multiple is, is about, is less than, is less than about, is more than, is more than about, 0.5, 1, 1.5, 2, 3, 4, 5, 6, 7, 8, 9, 10, 15, 20, 30, 40, 50, 60, 70, 80, 90, or 100, or a range defined by any two of these amounts.

The number of times the volume of blood being treated is required to be circulated through the device (treatment cycles) varies based on the replication rate of the virus being treated, the viral load or pfu/ml of the individual's blood, and the clearing rate of the device. The replication rate of viruses varies with each virus, but is known or can be determined by one skilled in the art. The viral load within the individual's blood is dictated by the replication rate of the virus less the clearance rate of the virus. Further, the percentage of virus within the organs (non-blood borne), and the level of infectivity of the individual being treated influence the viral load, but can generally be ascertainable by a skilled artisan. The clearing rate of a particular device, although usually fixed across a broad spectrum of viruses, can vary. The clearing rate of a particular device is ascertainable by a person of ordinary skill in the art. Accordingly, the clinically relevant number of circulations is ascertainable without undue experimentation. The term “therapeutically effective number of circulations,” as used herein, refers to the number of circulations determined by a person of ordinary skill in the art to reduce the pfu/ml or viral load of the blood by or to a therapeutically effective amount.

In some embodiments, the number of times the blood or plasma being treated, which can be equal to the total blood volume of the individual being treated, or a multiple thereof, circulates through the device is, is about, is less than, is less than about, is more than, is more than about 200, 175, 150, 125, 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, or 1. In some embodiments, the number of times the volume of blood equal to the total blood volume of the individual being treated circulates through the device is a range defined by any two numbers recited above.

Once the amount of blood or plasma to be processed and the number of circulations is determined, the time required for treatment is determined by the flow rate and capacity of the device. As such, the time required for a volume of blood or plasma to be processed on the device, or the amount of time an individual utilizes the device, can be determined by a skilled artisan. In some embodiments, the time required is, is about, is less than, is less than about, is more than, is more than about 1600 minutes, 1400 minutes, 1200 minutes, 1000 minutes, 800 minutes, 700 minutes, 600 minutes, 500 minutes, 490 minutes, 480 minutes, 470 minutes, 460 minutes, 450 minutes, 440 minutes, 430 minutes, 420 minutes, 410 minutes, 400 minutes, 390 minutes, 380 minutes, 370 minutes, 360 minutes, 350 minutes, 340 minutes, 330 minutes, 320 minutes, 310 minutes, 300 minutes, 290 minutes, 280 minutes, 270 minutes, 260 minutes, 250 minutes, 240 minutes, 230 minutes, 220 minutes, 210 minutes, 200 minutes, 190 minutes, 180 minutes, 170 minutes, 160 minutes, 150 minutes, 140 minutes, 130 minutes, 120 minutes, 110 minutes, 100 minutes, 90 minutes, 80 minutes, 70 minutes, 60 minutes, 50 minutes, 40 minutes, 30 minutes, 20 minutes, or 10 minutes. In other embodiments, the time required for an individual to be processed on the device is a range defined by any two times recited above. In preferred embodiments the individual can utilize the device while the individual is undergoing dialysis, such that use of the device and dialysis occur simultaneously. In such embodiments, the time an individual utilizes the device can be determined by the length of time taken to complete the dialysis session. Such time periods can be determined by the skilled artisan. In some embodiments, the individual's blood is continuously treated, and the device, or lectin portion of the device is periodically replaced.

In some embodiments, an individual can utilize the device in one or more sessions. A session can include the period of time for a volume of blood or plasma to be processed on the device as described herein. In some embodiments, an individual can utilize the device in at least 1 session, 5 sessions, 10 sessions, 50 sessions, 100 sessions, 500 sessions, 1000 sessions, 5000 sessions, or 10,000 sessions. In preferred embodiments, the individual can utilize the device while the individual is undergoing dialysis, such that use of the device and dialysis occur simultaneously.

The time period between consecutive sessions an individual utilizes the device can be more than or less than about 6 hours, 12 hours, 18 hours, 24 hours, 30 hours, 36 hours, or 42 hours, 3 days, 4 days, 5 days, 6 days, or 7 days, 1 week, 2 weeks, 3 weeks, 4 weeks, or 5 weeks, or 1 month, 2 months, 3 months, 4 months, 5 months, 6 months, 9 months, or 12 months. In preferred embodiments, the time period can be the time period between an individual's consecutive sessions utilizing a dialysis apparatus, where the dialysis apparatus can be utilized with the device simultaneously.

In some embodiments, the process reduces the viral load or pfu/ml in the blood or plasma by, by about, by at least, by at least about, by more than, by more than about 99.9%, 99.8%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65, 64%, 63%, 62%, 61%, 60%, 59%, 58%, 57%, 56%, 55%, 54%, 53%, 52%, 51%, 50%, 45%, 40%, 35%, 30%, 25%, 20%, 15%, or 10%. In some embodiments, the process reduces the viral load or pfu/ml in the blood or plasma by, by about, by at least, by at least about, by more than, by more than about a 1, 2, 3, 4, 5, 6, 7, 8, 9, or 10 log fold reduction. In other embodiments, the process reduces the viral load in the blood or plasma by a range defined by any two percentages recited above.

In some embodiments, the reduction in viral load or pfu/ml in an individual can occur within a limited amount of time. The amount of time required to reduce the viral load or pfu/ml to a desired level, or by a certain amount, is, is about, is less than, is less than about, is more than, is more than about 1600 minutes, 1400 minutes, 1200 minutes, 1000 minutes, 800 minutes, 700 minutes, 600 minutes, 500 minutes, 490 minutes, 480 minutes, 470 minutes, 460 minutes, 450 minutes, 440 minutes, 430 minutes, 420 minutes, 410 minutes, 400 minutes, 390 minutes, 380 minutes, 370 minutes, 360 minutes, 350 minutes, 340 minutes, 330 minutes, 320 minutes, 310 minutes, 300 minutes, 290 minutes, 280 minutes, 270 minutes, 260 minutes, 250 minutes, 240 minutes, 230 minutes, 220 minutes, 210 minutes, 200 minutes, 190 minutes, 180 minutes, 170, 160 minutes, 150 minutes, 140 minutes, 130 minutes, 120 minutes, 110 minutes, 100 minutes, 90 minutes, 80 minutes, 70 minutes, 60 minutes, 50 minutes, 40 minutes, 30 minutes, 20 minutes, or 10 minutes.

In certain embodiments, a reduction in viral load or pfu/ml in an individual may be observed subsequent to one or more sessions utilizing the device. The reduction in viral load or pfu/ml can be to a desired level, for example, a therapeutically effective amount or less. In some embodiments, an individual can utilize a device in at least 1 session, 2 sessions, 3 sessions, 4 sessions, 5 sessions, 10 sessions, 15 sessions, or 20 sessions before observing a reduction in viral load or pfu/ml to a therapeutically effective amount or less.

In some embodiments the devices and methods described herein preferentially remove live viral particles (pfu) from blood or plasma more readily than other viral particles or fragments thereof. In some embodiments, the ratio of percent pfu clearance to percent viral load clearance is, is about, is less than, is less than about, is more than, is more than about, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.5:1, 7.0:1, 7.5:1, 8.0:1, 8.5:1, 9.0:1, 9.5:1, 10:1, 15:1, 20:1, 30:1, 40:1, 50:1, 75:1, 100:1, 125:1, 150:1, 175:1, or 200:1, 400:1, 500:1, 750:1, or 1000:1. In other embodiments, the ratio of pfu clearance to viral load clearance is a range defined by any two ratios recited above.

In one embodiment, blood having viral particles and/or fragments thereof is withdrawn from a patient and contacted with a membrane. In one preferred embodiment, the blood is separated into its plasma and cellular components. The plasma is then contacted with the lectins to remove the viral particles or fragments thereof by binding between viral high mannose glycoproteins and lectins. The plasma can then be recombined with the cellular components and returned to the patient. Alternatively, the cellular components can be returned to the patient separately. The treatment can be repeated periodically until a desired response has been achieved.

The technology to immobilize enzymes, chelators, and antibodies in dialysis-like cartridges has been developed (Ambrus et al., Science 201(4358): 837-839, 1978; Ambrus et al., Ann Intern Med 106(4): 531-537, 1987; Kalghatgi et al. Res Commun Chem Pathol Pharmacol 27(3): 551-561, 1980) and is incorporated herein by reference. These cartridges can be directly perfused with blood from patients through direct venous access, and returned to the patients without further manipulations. Alternatively, blood can be separated into plasma and cellular components by standard techniques. The cellular components can be combined with the plasma before reinfusing or the cellular components can be reinfused separately. Viral load can be assessed in the effluent from the cartridge by standard techniques such as ELISA and nucleic acid amplification and detection techniques. Prototypic cartridges have been used to metabolize excess phenylalanine (Kalghatgi et al., 1980, supra; Ambrus, 1978, supra) or to remove excess aluminum from patients' blood (Anthone et al. J Amer Soc Nephrol 6: 1271-1277, 1995). An illustration of preparing proteins for immobilization to the hollow fibers for the method of the present invention is presented in U.S. Pat. Nos. 4,714,556 and 4,787,974, 5,528,057.

For binding of lectins to the membrane, the polymers of the membrane are first activated, i.e., made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3). Once the polymer has been activated, the lectins can be attached directly or via a linker to form in either case an affinity matrix. Suitable linkers include, but are not limited to, avidin, streptavidin, biotin, protein A, and protein G. The lectins can also be directly bound to the polymer of the membrane using coupling agents such as bifunctional reagents, or can be indirectly bound. In one embodiment, GNA covalently coupled to agarose can be used to form an affinity matrix.

In some embodiments, the lectin is attached to a substrate instead of, or in addition to, the membrane. Suitable substrates include, but are not limited to, silica (e.g. glass beads, sand, diatomaceous earth) polysaccharides (e.g. dextran, cellulose, agarose), proteins (e.g. gelatin) and plastics (e.g. polystyrenes, polysulfones, polyethersulfones, polyesters, polyurethanes, polyacrylates and their activated and native amino and carboxyl derivatives). The lectin can be bound to the substrates through standard chemical means, either directly, or through linkers such as C2 to C>20 linear and branched carbon chains, as well as the plastics, proteins and polysaccharides listed above. For most synthetic purposes, C18 is the preferred upper limit but the chains can be added together for solubility reasons. Preferred linkers include: C2 to C18 dicarboxylates, diamines, dialdehydes, dihalides, and mixtures thereof (e.g. aminocarboxylates) in both native and activated form (e.g. disuccinimidyl suberimidate (DSS)). In some embodiments, one or more substrates can be used as linkers, alone or in combination with the substances listed as linkers. For example, dextran can be attached to sand, and additional linkers can then optionally be added to the dextran.

Some embodiments include systems for reducing the viral load or pfu/ml in an individual to a desired level, for example, a therapeutically relevant amount. Such systems can include the device and a dialysis apparatus. Preferred embodiments include utilizing the device and a dialysis apparatus simultaneously.

Some embodiments include kits for reducing the viral load or pfu/ml in an individual to a desired level, for example, a therapeutically relevant amount. Such kits can include the device, instructions for use of the device, and one or more of each of the following: a tube, connector, valve, and coupler, or any combination thereof, for connecting the affinity cartridge to a dialysis apparatus. In one embodiment, the kit comprises a set of tubes, connectors and valves for connecting the device to a dialysis machine, as well as shunting the blood around the affinity cartridge as desired by the operator during dialysis. Use of the shunting connection allows for changing the affinity cartridge during dialysis without requiring stopping the dialysis procedure.

As used herein, individual, subject or patient, refers to any animal whose blood or other bodily fluid is being treated, and is not limited to humans. Individuals or subjects include all animals, including but not limited to primates such as monkeys and apes, dogs, cats, rats, mice, rabbits, pigs, and horses.

Although the embodiments described herein refer to removal of virus particles or fragments thereof from blood or plasma, one of skill in the art will appreciate that the device and methods described herein can be used with other fluids, such as other bodily fluids, cell culture supernatants, buffers, etc., which are contaminated with or contain lectin-binding virus or viral particles.

U.S. patent application Ser. No. 10/760,810, issued as U.S. Pat. No. 7,226,429, and the articles, patents and applications, and other printed materials referred to herein, are hereby incorporated by reference in their entirety, and particularly for the material referred to above.

The following examples are presented to illustrate embodiments of this invention and are not intended to be restrictive.

### EXAMPLES

### Example 1

### HCV Viral Load In Clinical Safety and Preliminary Efficacy Studies Objectives

Studies were performed to determine the safety of the Aethlon Medical GNA HEMOPURIFIER® in subjects who require kidney dialysis, namely, End Stage Renal Disease (ESRD) patients. Informed consent was obtained from the patients. Active components of the GNA HEMOPURIFIER® included Plasmart™ Plasma Separator—PS60, and Galanthus Nivalis Agglutinin (GNA).

Safety was evaluated during and after each treatment regimen using laboratory tests (hematology, clinical chemistry, liver function tests (LFTs)), clinical evaluations, and Karnofsky performance status to assess patient wellbeing and cardiovascular stability. In addition, blood samples were taken prior to treatment and at each time-point after treatment and tested to confirm the lack of GNA or silica leachables from the cartridge. Additionally, adverse event and hematology data was collected from ESRD patients undergoing standard of care intermittent dialysis for comparison to the same measurements in the same patients taken during treatment with the GNA HEMOPURIFIER® plus dialysis. Karnovsky scores, ECG, serologic and blood markers in each subject undergoing standard of care dialysis was compared to standard of care dialysis combined with the GNA HEMOPURIFIER® to identify any abnormal or significant deviations.

### Study Design

This was a single-arm, sequential, controlled study in which each subject served as his/her own control. Eligible subjects were treated for one week under standard of care intermittent dialysis conditions (three full dialysis sessions each lasting up to 4 hours) and baseline blood and adverse event parameters were measured. Subjects exhibiting cardiovascular stability, stable blood access fistulas and stable hematology were enrolled. On week two, patients received treatment with the GNA HEMOPURIFIER® three times per week coincident with their ongoing standard of care intermittent dialysis, for up to four hours per session, for one week. Subjects were assessed for cardiovascular stability, hematology, ECG and laboratory tests for one week following HEMOPURIFIER® treatment.

### Subject Eligibility

Eligible subjects were adults, aged 18 and older, with ESRD requiring dialysis. Eligible subjects were undergoing chronic intermittent dialysis via an arteriovenous fistula or graft. Subjects demonstrated cardiovascular stability (stable blood pressure, lack of cramps) for at least 1 month before entry into study with two to three full dialysis sessions per week each lasting 2 to 5 hours). The arteriovenous fistula/graft must have been in place for at least 1 month before entry into study. Ten study subjects were enrolled and divided between two study sites.

### Duration of Treatment

Qualified, consenting subjects were enrolled for one week of standard of care intermittent dialysis during which control assessments and data were collected. Study subjects then received one additional week of standard of care dialysis in addition to GNA HEMOPURIFIER® treatment. Standard of care intermittent dialysis was conducted for 2 to 5 hours per session, the length of which was determined by the physician investigator. In this phase 1 study the reference treatment was one week of standard of care intermittent dialysis (three full dialysis sessions each lasting up to 4 hours).

### Study Endpoints

All safety endpoints were assessed by comparison of results between treatment regimens. Primary endpoints included indications of: (1) Incidence and classification of unanticipated adverse device events during and at the end of HEMOPURIFIER® treatment. Adverse events were ranked by severity and relationship to the device; (2) Clinically significant changes in hematology during and after HEMOPURIFIER® treatment; (3) Clinically significant changes in clinical chemistry during and after HEMOPURIFIER® treatment; and (4) Evidence of significant elevation GNA or silica in the blood of patients after HEMOPURIFIER® treatment. Secondary endpoints included indications of: (1) Incidence and classification of adverse events; (2) Measurement of HCV viral load.

### Study Schedule

Consenting, study candidates had a medical history and physical examination, including performance status, clinical laboratory evaluations (including LFTs and electrocardiogram (ECG) prior their standard of care dialysis (control analysis) week. Study subjects were evaluated during three full standard of care dialysis sessions each lasting from 2-5 hours at the investigational physicians discretion. Following the control stage, each subject received a second week of standard of care intermittent dialysis treatment combined with the GNA Hemopurifier. During the control and treatment stages, evaluations, including adverse device effect monitoring, were conducted immediately before, during, and at varied times after each treatment. Each subject was evaluated during their follow on standard of care visit for one week following the last treatment, subjects received a physical examination, clinical laboratory evaluations (including LFTs) and ECG at each of three follow on visits over one week.

### Preliminary Results

Three patients have completed the study, providing preliminary safety and efficacy data. All three patients were on dialysis without the affinity capture device for at least 6 months prior to the study, receiving dialysis for about 4 hours a day on a Monday, Wednesday, Friday, or Tuesday, Thursday, Saturday schedule. During the study, the subjects received dialysis alone on days 1, 3, and 5, followed by dialysis plus the affinity capture device on days 8, 10, and 12, followed by dialysis alone on days 15, 17, and 19. Samples of blood were taken prior to treatment on days 1, 3, 5, 8, 10, 12, 15 and 19. The total HCV viral load of each sample was assessed using RT-PCR. Control samples were measured in duplicate while treatment samples were generally measured in triplicate.

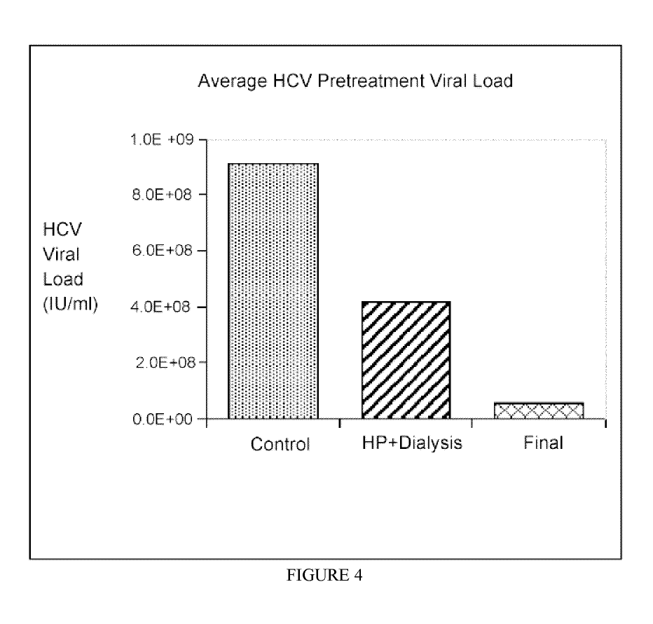

The resulting data documented that two of three HCV patients tested responded with measurable viral load reductions during the course of three 4-hour HEMOPURIFIER® plus dialysis treatments. The third patient showed both increases and decreases in viral load during the course of treatment, but demonstrated an overall reduction in follow-on viral load tests (days 15 and 19). Given the small sample size, viral load data was averaged for all 3 patients. Average initial HCV viral load was 3.13×10⁸ viral units per ml of blood. After completion of three HEMOPURIFIER® plus dialysis treatments, viral load was reduced an average 57% (final 4.1×10⁷ IU/ml). The stepwise drop in HCV viral load averaged 36% per treatment. Follow-on testing indicated that HCV viral load was 60% lower than initial viral load values when measured three days after final HEMOPURIFIER® plus dialysis treatment (day 15), and at seven days post treatment (day 19) viral load declined to 82% below starting viral load values. FIG. 4 shows the average of the viral load for the samples taken on days 1, 3, 5 and 8 (Control), on days 10, 12, and 15 (HP+dialysis), and day 19 (Final, average of two patients). These results show that treatment with the HEMOPURIFIER® plus dialysis lowered the average viral load compared to average viral load with dialysis alone. Surprisingly, after only three treatments with the HEMOPURIFIER®, the reduction in viral load was sustained for at least one week as shown in the Final group. None of the patients were being treated with antiviral drug therapy. In sum, the HEMOPURIFIER® treatment of HCV infected patients undergoing dialysis resulted in a net viral load reduction of 60 to 80% with the effects of treatment progressing at least 7 days beyond HEMOPURIFIER® treatment.

While some literature discloses that kidney dialysis alone can transiently reduce HCV viral load, this effect is typically short-lived, with the viral load typically returning to pretreatment levels prior to the next dialysis, which is typically 1-3 days later. Without being bound by any theory, it is believed that dialysis alone may remove predominantly non-infectious virus or fragments, which represent 99% of the total viral load, presumably because the pore size of the dialysis fibers is too small (about 4 nm) to allow whole infectious HCV virus (about 50-100 nm) to pass. In contrast, the HEMOPURIFIER® is designed with a pore size sufficient to allow both fragments and whole viral particles to pass, and be captured on the affinity matrix. It is believed that removal of infectious viral particles by the HEMOPURIFIER® treatment in addition to dialysis results in reduced infection and viral replication, and ultimately viral load. This effect can be sustained for at least a week following as few as three HEMOPURIFIER® treatments. Further reductions in viral load are expected with additional HEMOPURIFIER® treatments.

The above description discloses subject matter including several embodiments of compositions, methods, and systems. This subject matter is susceptible to modification, and such modifications will become apparent to those skilled in the art from a consideration of this description and/or practice of the embodiments disclosed herein. Consequently, it is not intended that this invention be limited to the specific embodiments disclosed herein, but that it cover all modifications and alternatives coming within the true scope and spirit of the invention.

All references cited herein including, but not limited to, published and unpublished applications, patents, literature references and web-sites, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

What is claimed is:

1. A method for producing a sustained reduction in the viral load of Hepatitis C virus (HCV) in blood from a patient undergoing hemodialysis comprising:
(a) identifying a patient infected with HCV in need of hemodialysis;
(b) removing blood from said patient;
(c) transferring said blood or a portion thereof to a lectin affinity device comprising:
(i) a processing chamber configured to receive said blood or portion thereof, said chamber having lectin disposed therein such that said lectin binds HCV viral particles in said blood or portion thereof, and wherein said device is configured to retain said lectin and bound HCV viral particles in said chamber, and
(ii) a porous membrane configured to permit passage of viral particles through the pores of said membrane such that said viral particles contact said lectin and said membrane excludes substantially all blood cells from passing through said pores, such that said blood cells do not contact said lectin,
wherein said transferring further comprises transferring said blood or portion thereof to said chamber such that said HCV viral particles contact said lectin and are bound thereto;
(d) removing said blood or portion thereof from said chamber, and
(e) returning said blood or portion thereof to said patient.

2. The method of claim 1, further comprising repeating steps (b)-(e).

3. The method of claim 2, wherein said method is sufficient to reduce at least 10% of the HVC viral load in a sample of blood from said patient.

4. The method of claim 2, wherein said method is sufficient to reduce at least 50% of the HCV viral load in a sample of blood from said patient.

5. The method of claim 2, wherein said method is sufficient to reduce the HCV viral load in a sample of blood from said patient to no greater than about 4.1×10⁷ copies/ml of HCV RNA.

6. The method of claim 2, wherein said method is performed until a volume of blood approximately equal to the total blood volume of said patient has been exposed to said lectin.

7. The method of claim 1, wherein a reduction in viral load is sustained in said patient for at least about 7 days.

8. The method claim 1, wherein said membrane comprises one or more porous hollow fiber membranes, wherein lectin is disposed within an extralumenal space of said chamber proximate to an exterior surface of said membranes, wherein the lectin binds said viral particles and traps them in said extralumenal space, and wherein step (c) further comprises: passing said blood or portion thereof through said hollow fiber membranes, and collecting the pass-through blood or portion thereof.

9. The method of claim 8, wherein said membrane comprises pores less than about 700 nm in diameter.

10. The method of claim 8, wherein said membrane comprises an inside diameter of about 0.3 mm and an outside diameter of about 0.5 mm.

11. The method of claim 1, further comprising passing said blood or portion thereof through a hemodialysis apparatus.

12. The method of claim 8, further comprising passing said blood or portion thereof through a hemodialysis apparatus.

13. The method of claim 1, wherein said blood or portion thereof is exposed to said lectin for no longer than 360 minutes.

14. The method of claim 1, wherein said method is repeated for about 1 to 6 hours a day, for at least 3 days a week, wherein said method is discontinued after a period of time not less than about 1 week, wherein said method is sufficient to reduce said viral load by at least about 10% for a period of at least about 1 week following the discontinuation of said method.

15. The method of claim 1, wherein said patient comprises an End Stage Renal Disease (ESRD) patient.

16. A system for producing a sustained reduction in the viral load of Hepatitis C virus (HCV) in blood from a hemodialysis patient infected with HVC comprising:
a lectin affinity device comprising a processing chamber having lectin disposed within said processing chamber, wherein said lectin binds viral particles in said blood and traps said viral particles in said processing chamber, and a porous membrane configured to permit passage of viral particles through the pores of said membrane such that said viral particles contact said lectin and said membrane excludes substantially all blood cells from passing through said pores, such that said blood cells do not contact said lectin; and
a hemodialysis apparatus.

17. The system of claim 16, wherein said lectin affinity device and said hemodialysis apparatus are configured to permit simultaneous use on an patient.

18. The system of claim 16, wherein said lectin affinity device and said hemodialysis apparatus are connected to establish an extracorporeal circulation system with said patient.

19. The system of claim 18, wherein said lectin affinity device and said hemodialysis apparatus are connected in series.

20. An improved hemodialysis method for patient's undergoing hemodialysis who are infected with HCV, said improvement comprising passing said patient's blood through an lectin affinity device during said hemodialysis, wherein said lectin affinity device comprises a processing chamber having lectin disposed within said processing chamber, wherein said lectin binds HCV viral particles in said blood, and a porous membrane configured to permit passage of viral particles through the pores of said membrane such that said viral particles contact said lectin and said membrane excludes substantially all blood cells from passing through said pores, such that said blood cells do not contact said lectin, and wherein said device is configured to retain said lectin and bound HCV viral particles in said processing chamber.
